(19) United States
(12) Patent Application Publication
(10) Pub. No.: US 2016/0081704 A1
(43) Pub. Date: Mar. 24, 2016

(54) CATHETER FOR EXTRACTION OF FOREIGN SUBSTANCE FROM BLOOD VESSEL

(71) Applicant: THEINGMEDICAL CO., LTD., Okcheon-gun (JP)

(72) Inventors: Eui Yong Jeon, Seoul (KR); Jae Hwang Jeong, Cheongwon-gun (KR); Jeong Kwon Lee, Seongnam (KR); Jae Hyun Park, Pyeongtaek (KR)

(21) Appl. No.: 14/954,448

(22) Filed: Nov. 30, 2015

Related U.S. Application Data
(63) Continuation of application No. PCT/KR2014/004726, filed on May 28, 2014.

(30) Foreign Application Priority Data
May 31, 2013 (KR) 10-2013-0062784

(51) Int. Cl. A61B 17/221; A61B 17/32

(52) U.S. Cl. CPC A61B 17/221; A61B 17/32002; A61B 2017/22079

(57) ABSTRACT

Disclosed is a catheter for the extraction of foreign substances from blood vessels including an insertion tube configured to be inserted into a blood vessel and provided at one side thereof with a branched suction tube, a moving crusher configured to be inserted into the blood vessel while being received inside the insertion tube, the moving crusher serving to crush foreign substances inside the blood vessel after being discharged out of the insertion tube, and a foreign substance moving net coupled to a front end of the moving crusher so as to guide the crushed foreign substances into the insertion tube, the foreign substance moving net having a mesh shape. The foreign substance moving net includes an introduction net having an open front end to allow the foreign substances to be introduced thereinto, a flow net connected to the introduction net and configured to provide a passage for movement of the foreign substances, and a discharge net configured to couple the flow net and the moving crusher to each other and to discharge the foreign substances into the insertion tube, and the flow net is provided at a surface thereof with a coating layer to prevent the foreign substances from being discharged out of the flow net.

### CROSS-REFERENCE TO RELATED APPLICATIONS

The present application is a continuation application of International Application No. PCT/KR2014/004726 filed on May 28, 2014, which claims priority to Korean Application No. 10-2013-0062784 filed on May 31, 2013. The applications are incorporated herein by reference.

### TECHNICAL FIELD

The present invention relates to a catheter for the extraction of foreign substances from blood vessels and, more particularly, to a catheter for the extraction of foreign substances from blood vessels, which is capable of crushing foreign substances present inside blood vessels, and subsequently effectively extracting the crushed foreign substances out of the blood vessels.

### BACKGROUND ART

In general, catheters for medical procedures are used to support the discharge of content solutions from, for example, body cavities and tubular and chambered organs. Extraction catheters contribute to the improvement of blood flow through the insertion of a tube into a blood vessel such as, for example, an arterial, venous, or cerebrovascular blood vessel, as well as the aforementioned organs, after which a separator is inserted through the tube to crush foreign substances such as, for example, blood clots present in the blood vessel, after which the crushed foreign substances are suctioned and finally removed through the inserted tube.

In relation to this, Korean Patent Laid-Open Publication No. 2012-0010927 discloses “Catheter for Extraction of Foreign Substance from Blood Vessel”.

However, in the disclosed conventional extraction catheter, a separator, which is introduced or discharged through a tube inserted into a blood vessel and is used to crush foreign substances, has, for example, hook-shaped portions, linear portions, and curvilinear portions. As such, crushed foreign substances may not be wholly discharged out of the blood vessel, but may be deposited in the blood vessel again, which requires the extraction of the foreign substances to be repeatedly conducted.

### SUMMARY

One object of the present invention is to provide a catheter for the extraction of foreign substances from blood vessels, which is capable of extracting all of crushed foreign substances out of a blood vessel.

Another object of the present invention is to provide a catheter for the extraction of foreign substances from blood vessels, which is capable of preventing foreign substances from being discharged out of the catheter while moving through the catheter, thereby improving the efficiency with which the foreign substances is discharged.

The objects described above and several advantages of the present invention will be more clearly understood from exemplary embodiments of the present invention by those skilled in the art.

The objects of the present invention may be accomplished by a catheter for the extraction of foreign substances from a blood vessel. The catheter of the present invention includes an insertion tube configured to be inserted into a blood vessel and provided at one side thereof with a branched suction tube, a moving crusher configured to be inserted into the blood vessel while being received inside the insertion tube, the moving crusher serving to crush foreign substances inside the blood vessel after being discharged out of the insertion tube, and a foreign substance moving net coupled to a front end of the moving crusher so as to guide the crushed foreign substances into the insertion tube, the foreign substance moving net having a mesh shape, wherein the foreign substance moving net includes an introduction net having an open front end to allow the foreign substances to be introduced thereinto, a flow net connected to the introduction net and configured to provide a passage for movement of the foreign substances, and a discharge net configured to couple the flow net and the moving crusher to each other and to discharge the foreign substances into the insertion tube, and wherein the flow net is provided at a surface thereof with a coating layer to prevent the foreign substances from being discharged out of the flow net.

According to one embodiment, the coating layer may be formed of silicon or PTFE. According to one embodiment, the moving crusher may include a moving shaft configured to move inside the insertion tube, and a pressure tip located at a front end of the moving shaft and configured to apply pressure to the foreign substances so as to crush the foreign substances, and the foreign substance moving net may be coupled to the moving shaft at a rear end of the pressure tip.

According to one embodiment, the discharge net may be gradually reduced in diameter in a direction in which the discharge net is coupled to the moving shaft, and may guide the foreign substances so as to be discharged between the moving shaft and the insertion tube.

Meanwhile, the objects of the present invention may be accomplished by a catheter for the extraction of foreign substances from blood vessels. The catheter for the extraction of foreign substances from blood vessels may include an insertion tube configured to be inserted into a blood vessel and provided at one side thereof with a branched foreign substance discharge tube, a moving crusher configured to be inserted into the blood vessel while being received inside the insertion tube, the moving crusher serving to crush foreign substances inside the blood vessel after being discharged out of the insertion tube, a foreign substance moving net coupled to a front end of the moving crusher so as to guide the crushed foreign substances into the insertion tube, the foreign substance moving net having a tubal shape such that a diameter thereof is gradually reduced with increasing distance from an open front end thereof and decreasing distance to a rear end thereof coupled to the moving crusher, and a pressure wire having one end secured to an inner sidewall of the front end of the foreign substance moving net and the other end extending outward from the foreign substance moving net so as to change the shape of the foreign substance moving net by applying pressure to the front end of the foreign substance moving net, wherein the foreign substance moving net includes an introduction net having an open front end to allow the foreign substances to be introduced thereinto, a flow net connected to the introduction net and configured to provide a passage for movement of the foreign substances, and a discharge net configured to couple the flow net and the moving crusher to each other and to discharge the foreign substances into the insertion tube, and wherein the flow net is provided at a surface thereof with a coating layer to prevent the foreign substances from being discharged out of the flow net.

According to one embodiment, the moving crusher may include a moving shaft configured to move inside the insertion tube, and a pressure tip located at a front end of the moving shaft and configured to apply pressure to the foreign substances so as to crush the foreign substances, and the foreign substance moving net may be coupled to the moving shaft at a rear end of the pressure tip. According to one embodiment, the catheter may further include a wire winder, on which the other end of the wire is wound such that a user applies pressure to a front end of the wire by winding or unwinding the wire.

A catheter for the extraction of foreign substances from blood vessels according to the present invention is capable of effectively crushing foreign substances using pressure applied by a pressure tip. In addition, most of the crushed foreign substances may be introduced into an insertion tube by a foreign substance moving net.

In addition, the surface of the foreign substance moving net is coated, so as to ensure that the foreign substances introduced into the foreign substance moving net are completely extracted out of a blood vessel, rather than being discharged into the blood vessel through the foreign substance moving net.

Accordingly, the extraction efficiency may be enhanced, which may reduce the duration of a medical procedure.

In addition, in the catheter for the extraction of foreign substances from blood vessels according to the present invention, the shape and diameter of the foreign substance moving net may be changed by pressure applied from a pressure wire. As such, the shape of the foreign substance moving net may be changed during use to correspond to the diameter of the blood vessel into which the foreign substance moving net is inserted.

### BRIEF DESCRIPTION OF DRAWINGS

FIG. 1 is a perspective view illustrating the configuration of a catheter for the extraction of foreign substances from blood vessels according to an exemplary embodiment of the present invention,

FIG. 2 is a view illustrating the process of extracting foreign substances using the catheter for the extraction of foreign substances from blood vessels inserted into a blood vessel according to the exemplary embodiment of the present invention,

FIG. 3 is an enlarged view illustrating the configuration of a foreign substance moving net included in the catheter for the extraction of foreign substances from blood vessels according to the exemplary embodiment of the present invention,

FIG. 4 is a perspective view illustrating the configuration of a catheter for the extraction of foreign substances from blood vessels according to another exemplary embodiment of the present invention,

FIG. 5 is a view illustrating variation in the shape of a foreign substance moving net included in the catheter for the extraction of foreign substances from blood vessels according to another exemplary embodiment of the present invention,

FIG. 6 is a view illustrating the process of extracting foreign substances using the catheter for the extraction of foreign substances from blood vessels inserted into a blood vessel according to another exemplary embodiment of the present invention, and

FIG. 7 is an enlarged view illustrating the configuration of a foreign substance moving net included in the catheter for the extraction of foreign substances from blood vessels according to another exemplary embodiment of the present invention.

### DESCRIPTION OF REFERENCE NUMERALS

100: catheter for the extraction of foreign substances from blood vessels

110: insertion tube

111: insertion tube main body

113: suction tube

114: front end

115: rear end

120: moving crusher

121: moving shaft

123: pressure tip

123a: front end of the tip

125: torque piece

130: foreign substance moving net

131: introduction net

133: flow net

135: discharge net

135a: shaft coupling end

140: suction unit

200: catheter for the extraction of foreign substances from blood vessels

210: insertion tube

211: insertion tube main body

213: suction tube

214: front end

215: rear end

220: moving crusher

221: moving shaft

223: pressure tip

223a: front end of the tip

225: torque piece

230: foreign substance moving net

231: introduction net

233: flow net

235: discharge net

235a: shaft coupling end

240: suction unit

250: pressure wire

260: wire winder

261: support plate

263: rotating shaft

265: handle

### DETAILED DESCRIPTION

For sufficient understanding of the present invention, exemplary embodiments of the present invention will be described with reference to the accompanying drawings. The embodiments of the present invention may be modified into various forms, and the scope of the present invention should not be construed as being limited to the embodiments that will be described below in detail. The present embodiments are provided to provide one of ordinary skill in the art with a more complete description of the present invention. Accordingly, in the drawings, for example, the shapes of components may be exaggerated for emphasis and to clarify the description. It should be noted that the same members are shown by the same reference numerals throughout the respective drawings. A detailed description of known functions and configurations will be omitted when it may make the subject matter of the present invention rather unclear.

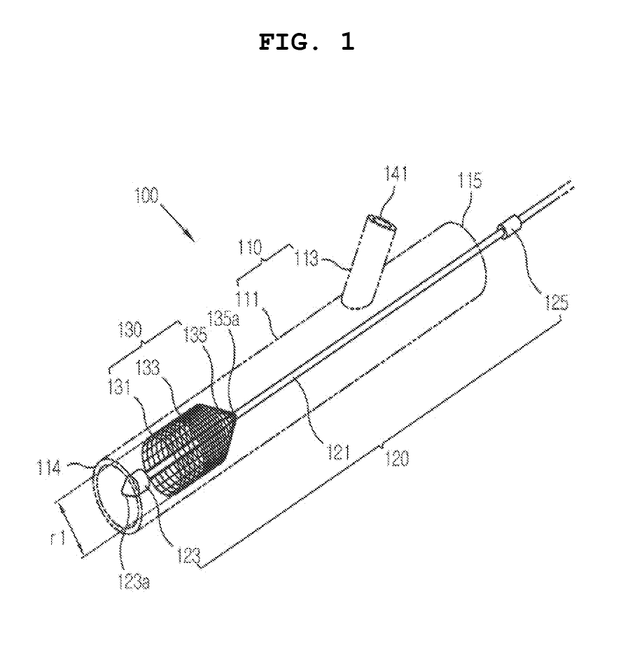

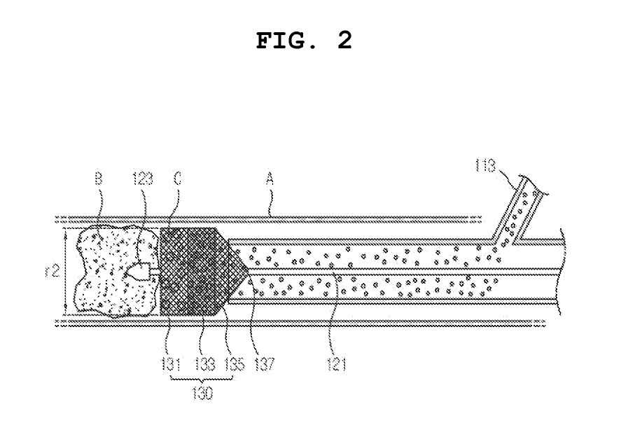

FIG. 1 is a perspective view illustrating the configuration of a catheter 100 for the extraction of foreign substances from blood vessels according to the present invention, and FIG. 2 is a view illustrating the process of extracting foreign substances using the catheter 100 for the extraction of foreign substances from blood vessels inserted into a blood vessel according to the present invention.

As illustrated, the catheter 100 for the extraction of foreign substances from blood vessels according to the present invention includes an insertion tube 110 which is configured to be inserted into a blood vessel, a moving crusher 120 which is received and moved inside the insertion tube 110 and crushes foreign substances, and a foreign substance moving net 130 which is coupled to a front end of the moving crusher 120 and guides the crushed foreign substances into the insertion tube 110.

The insertion tube 110 is configured as a hollow tube having a flow passage therein, through which the moving crusher 120 is movable. A front end 114 of the insertion tube 110 is open, so as to expel the moving crusher 120 into the blood vessel.

A rear end 115 of the insertion tube 110 is also open, so as to allow the operator to insert and move the moving crusher 120 through the rear end 115. A suction tube 113 branches from an outer circumferential surface of a rear end portion of the insertion tube 110. A suction unit 140, such as a pump, is coupled to the suction tube 113 and provides suction force required to extract the foreign substances out of the blood vessel. Due to the suction force provided from the suction unit 140, the foreign substances are moved through the interior of the insertion tube 110 to thereby be extracted out of the blood vessel through the suction tube 113.

The suction tube 113 and the rear end 115 of the insertion tube 110 are located outside the blood vessel, and the front end 114 of the insertion tube 110 is inserted into the blood vessel.

The insertion tube 110 may be provided in various diameters according to the kind and diameter of the blood vessels A from which foreign substances will be removed.

The insertion tube 110 is formed of a flexible material having good elasticity to ensure insertion thereof into the blood vessel. As such, after being inserted into the blood vessel, the insertion tube may be effectively moved along a blood vessel having an irregular path such as, for example, a curvilinear path, to an occluded location at which blood clots and foreign substances are present.

The moving crusher 120 is moved through the interior of the insertion tube 110 by external force provided by the operator, and crushes the foreign substances inside the blood vessel. The moving crusher 120 includes a moving shaft 121 which is inserted into the insertion tube 110, a pressure tip 123 which is coupled to a front end of the moving shaft 121 and crushes the foreign substances, and a torque piece 125 which is coupled to a rear end of the moving shaft 121 and is configured to receive force applied by the operator.

The moving shaft 121 is moved through the interior of the insertion tube 110 when the operator applies external force through the torque piece 125, thereby adjusting the position of the pressure tip 123. The moving shaft 121 may take the form of a wire having sufficient strength to be moved freely forward and rearward by the external force. The pressure tip 123 is located at the front end of the moving shaft 121, and comes into contact with a foreign substances B so as to apply pressure to the foreign substances B via movement of the moving shaft 121, thereby crushing the foreign substances B into small fragments. The diameter of the pressure tip 123 is gradually reduced with decreasing distance to a front end thereof. As such, a front end 123a of the tip has a pointed shape. This may ensure the effective and rapid crushing of blood clots and the foreign substances B as pressure is applied to the foreign substance.

The pressure tip 123 may be formed of a hard material, in order to apply sufficient pressure to the foreign substances.

The torque piece 125 is coupled to the rear end of the moving shaft 121 and is configured to allow the operator to apply external force by gripping the torque piece in the hand. Since the operator cannot easily grip the moving shaft 121 in the form of a thin wire in the hand and apply external force thereto, the torque piece 125 is separately coupled to an outer circumference of the moving shaft 121 so as to be easily gripped by the operator.

The foreign substance moving net 130 is coupled to the front end of the moving crusher 120, and guides the foreign substances and blood clots, crushed by the pressure tip 123, into the insertion tube 110. The foreign substance moving net 130 is formed into a mesh shape and is elastically expandable or contractible in volume. As such, in a state in which the moving crusher 120 is received in the insertion tube 110, the foreign substance moving net 130 remains contracted so as to conform to the inner diameter of the insertion tube 110, and as exemplarily illustrated in FIG. 2, expands after being discharged out of the insertion tube 110.

The foreign substance moving net 130 includes an introduction net 131 which is open toward the blood vessel A such that the foreign substances B are introduced into the introduction net 131, a discharge net 135 which is coupled to the moving shaft 121, and a flow net 133 which connects the introduction net 131 and the discharge net 135 to each other so as to define a foreign substance movement path.

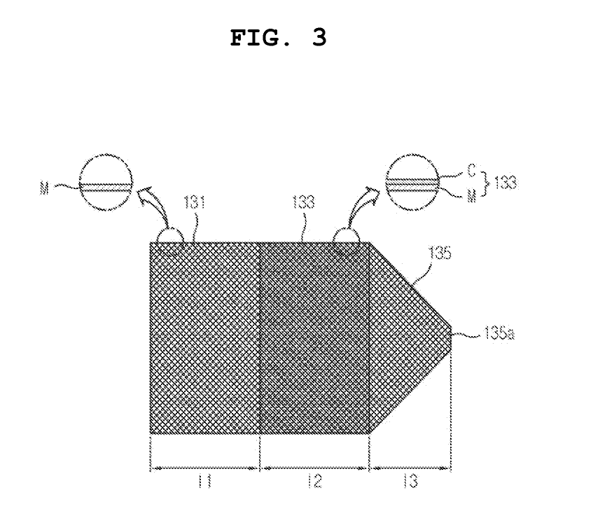

FIG. 3 is an enlarged view illustrating the configuration of the foreign substance moving net 130.

As illustrated, the entire foreign substance moving net 130 is formed of a mesh material M in a lattice shape or a spiral shape. The mesh material M has mesh holes formed in a surface thereof. Since the introduction net 131 is open toward the blood vessel A, the foreign substances and blood clots may be introduced into the foreign substance moving net 130 through the opening in the front end of the introduction net 131. In addition, the foreign substances and blood clots may also be introduced through the mesh holes formed in the surface of the mesh material M.

The flow net 133 is connected to a rear end of the introduction net 131 and moves the introduced foreign substances B into the discharge net 135. At this time, since suction force from the suction unit 140 is applied to the interior of the insertion tube 110, the foreign substances B is moved to the rear end by the suction force.

The flow net 133 may be formed of the same mesh material M as the introduction net 131, or may additionally have a coating layer c formed on the surface thereof. The coating layer c may be applied on the outer side of the mesh material M, and may prevent the introduced foreign substances and blood clots from being discharged out of the foreign substance moving net 130. The coating layer c may be formed of, for example, silicon or PTFE.

The thickness of the coating layer c may be determined such that it covers only the mesh holes in the surface of the mesh material M. The discharge net 135 extends from a rear end of the flow net 133 and is coupled to the moving shaft 121. The discharge net 135 discharges the foreign substances, moving through the flow net 133, into the insertion tube 110. The diameter of the discharge net 135 is gradually reduced in toward a rear end thereof. A shaft coupling end 135a formed at the rear end of the discharge net 135 is secured to the moving shaft 121. The shaft coupling end 135a is secured to the moving shaft 121 by, for example, an adhesive or thermal fusion. As such, the foreign substance moving net 130 is secured to the moving crusher 120 and moves into or out of the insertion tube 110 in linkage with the movement of the moving crusher 120.

The discharge net 135 is formed into a mesh shape in the same manner as the introduction net 131. Thus, the foreign substances moving through the flow net 133 are discharged into the insertion tube 110 through the discharge net 135.

Here, the length 11 of the introduction net 131, the length 12 of the flow net 133, and the length 13 of the discharge net 135 may be controlled in different manners according to the diameter and kind of the blood vessel A.

In addition, when the operator operates the moving crusher 120, the introduction net 131 and the flow net 133 may be discharged from the insertion tube 110 and the discharge net 135 may be disposed inside the insertion tube 110, which may prevent the outward leakage of the foreign substances to the maximum extent and may ensure the outward extraction of all of the crushed foreign substances. The process of operating the catheter 100 for the extraction of foreign substances from blood vessels according to the present invention having the above-described configuration will be described with reference to FIGS. 1 to 3.

The catheter 100, as exemplarily illustrated in FIG. 1, is distributed in a state in which the moving crusher 120 and the foreign substance moving net 130 are received inside the insertion tube 110. In this state, the catheter 100 is packaged and distributed in a hermetically sealed state, and is supplied to, for example, a hospital. The operator separates a wrapper therefrom for use.

The operator couples the suction unit 140, such as a pump, to the suction tube 113, and inserts the front end 114 of the insertion tube 110 into the blood vessel A. The insertion tube 110 is formed of a flexible material, and thus may be easily inserted into the blood vessel.

Simultaneously with the insertion of the insertion tube 110, the moving crusher 120 and the foreign substance moving net 130, received in the insertion tube, are moved to the target position at which the foreign substances are crushed. When the front end of the insertion tube 110 is moved to the target position, the operator discharges the moving crusher 120 out of the insertion tube 110 using the torque piece 125. As a result, the extruded foreign substance moving net 130 expands.

As the operator moves the moving shaft 121 back and forth, the pressure tip 123 crushes the blood clots and foreign substances B. A lump of crushed foreign substances C is introduced into the introduction net 131 and is then moved to the flow net 133. At this time, since the surface of the flow net 133 is covered with the coating layer c, the lump of crushed foreign substances C is moved through the flow net 133, rather than being discharged out of the flow net 133.

In addition, the lump of crushed foreign substances is discharged into the insertion tube 110 through the discharge net 135, and is drawn by suction force so as to be extracted into the suction tube 113.

The operator continuously applies pressure to the foreign substances B so as to crush the same while moving the moving shaft 121 back and forth, and the foreign substance moving net 130 expands and contracts by repeatedly entering and exiting the insertion tube 110 through the front end of the insertion tube 110. Thereby, most of the lump of crushed foreign substances C is extracted out of the blood vessel through the foreign substance moving net 130. As described above, the catheter for the extraction of foreign substances from blood vessels according to the present invention may effectively crush the foreign substances by applying pressure thereto using the pressure tip. In addition, most of the crushed foreign substances may be introduced into the insertion tube by the foreign substance moving net.

In addition, by coating the surface of the flow net of the foreign substance moving net, the foreign substances introduced into the insertion tube may be completely extracted out of the blood vessel, rather than being discharged into the blood vessel.

Accordingly, the extraction efficiency may be increased, which results in a reduction in the duration of a procedure.

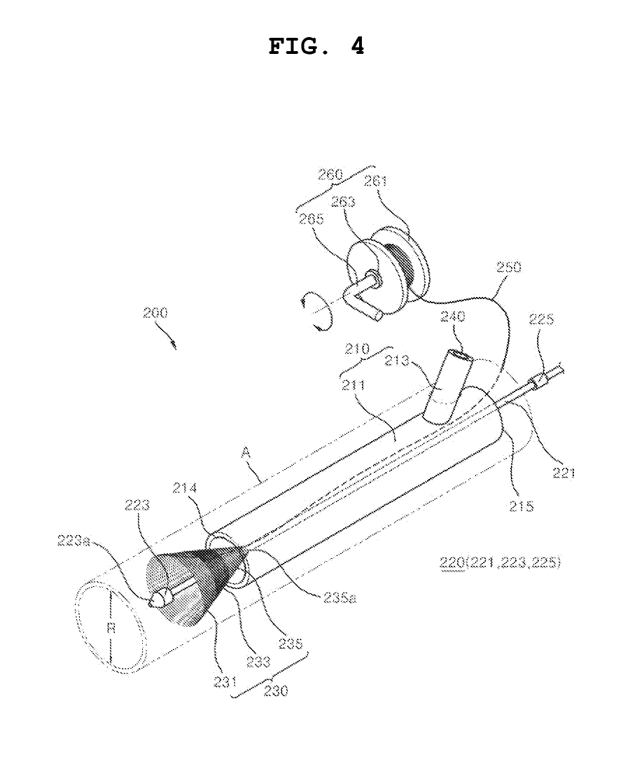

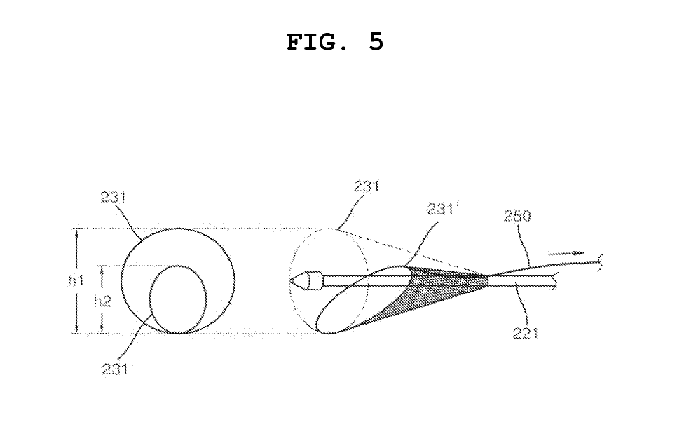

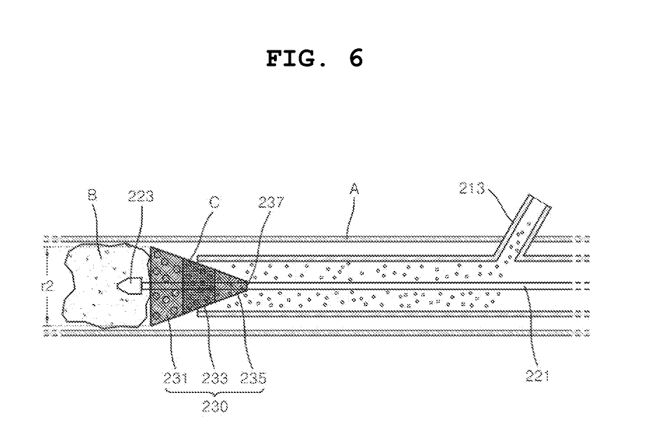

Meanwhile, FIGS. 4 to 7 are views illustrating the configuration of a catheter 200 for the extraction of foreign substances from blood vessels according to another embodiment of the present invention. FIG. 4 is a perspective view illustrating the configuration of a catheter 200 for the extraction of foreign substances from blood vessels according to the present invention, and FIG. 5 is a view illustrating the process of extracting foreign substances using the catheter 200 for the extraction of foreign substances from blood vessels inserted into a blood vessel according to the present invention.

As illustrated, the catheter 200 for the extraction of foreign substances from blood vessels according to the present invention includes an insertion tube 210 which is inserted into a blood vessel, a moving crusher 220 which is moved while being received inside the insertion tube 210 and crushes foreign substances, a foreign substance moving net 230 which has a tubal shape and is coupled to a front end of the moving crusher 220 so as to guide the crushed foreign substances into the insertion tube 210, a pressure wire 250 which is coupled to a front end of the foreign substance moving net 230 and applies pressure to the foreign substance moving net 230, and a wire winder 260 which adjusts tension of the pressure wire 250.

The insertion tube 210 and the moving crusher 220 have the same configuration as the insertion tube 110 and the moving crusher 220 of the catheter 100 for the extraction of foreign substances from blood vessels according to the previously described exemplary embodiment, and thus a detailed description thereof will be omitted.

The foreign substance moving net 230 is coupled to the front end of the moving crusher 220 and guides the foreign substances and blood clots, crushed by a pressure tip 223, into the insertion tube 210.

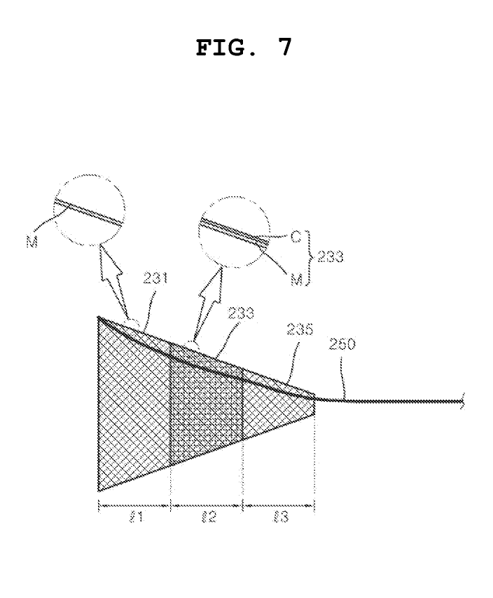

FIG. 7 is an enlarged view illustrating the configuration of the foreign substance moving net 230. Compared to the foreign substance moving net 130 of the catheter 100 for the extraction of foreign substances from blood vessels according to the previously described exemplary embodiment, the foreign substance moving net 230 has the same function, but differs in terms of the overall shape. Although the moving net 133 and the introduction net 131 of the foreign substance moving net 130 have the same diameter, an introduction net 231, a flow net 233, and a discharge net 235, which constitute the foreign substance moving net 230, are generally tapered into a tubal shape. The pressure wire 250, as exemplary illustrated in

FIG. 7, extends outward from a front end of the introduction net 231, and causes variation in the shape of the foreign substance moving net 230. One end of the pressure wire 250 is fixed to the front end of the introduction net 231, and the other end is coupled to the wire winder 260. When tension is applied to the pressure wire 250 as the user winds the pressure wire 250, the front end of the foreign substance moving net 230 is pulled to a moving shaft 221. Thereby, as exemplarily illustrated in FIG. 5, the shape of the foreign substance moving net 230 is changed. That is, in a loose state in which no pressure is applied to the pressure wire 250, the introduction net 231 has a circular shape and a diameter h1. Then, when pressure is applied to the pressure wire 250, the introduction net 231 is pulled rearward such that the front end of the introduction net 231 has an oval shape. At this time, the diameter h2 of the introduction net is reduced compared to the diameter h1.

Accordingly, when the diameter R of the blood vessel into which the catheter 200 will be inserted is large, the user may move only the moving shaft 221 back and forth, without applying pressure to the pressure wire 250. When the diameter R of the blood vessel is small, the user may apply pressure to the pressure wire 250 by winding the pressure wire, so as to reduce the foreign substance moving net 230 to the diameter h2.

Here, since the foreign substance moving net 230 is formed of a mesh material, the foreign substance moving net 230 naturally returns to its original diameter R1 when the pressure applied to the pressure wire 250 is reduced.

The pressure wire 250 may take the form of a nitinol wire, which may be used in blood vessels. The wire winder 260 winds the rear end of the pressure wire 250 so as to apply tension to the pressure wire 250. The wire winder 260 is used to allow the user to easily apply pressure to the pressure wire 250. The wire winder 260 includes a rotating shaft 263 around which the pressure wire 250 is wound, support plates 261 configured to support both ends of the rotating shaft 263, and a handle 265 configured to rotate the rotating shaft 263.

The user can wind the pressure wire 250 to apply pressure to the foreign substance moving net 230 so as to reduce the diameter thereof when rotating the handle 265 in a given direction, or may unwind the pressure wire 250 so as to return the foreign substance moving net 230 to its initial state by rotating the handle 265 in the opposite direction.

The process of operating the catheter 200 for the extraction of foreign substances from blood vessels according to another embodiment of the present invention having the above-described configuration will be described with reference to FIGS. 4 to 7.

The operator couples a suction unit 240, such as a pump, to a suction tube 213, and inserts a front end 214 of the insertion tube 210 into the blood vessel A. The insertion tube 210 is formed of a flexible material, and thus may be easily inserted into the blood vessel. At this time, the pressure wire 250 and the wire winder 260 may be coupled by the user, or may be provided in a previously coupled state.

Simultaneously with the insertion of the insertion tube 210, the moving crusher 220 and the foreign substance moving net 230, received in the insertion tube, are moved to the target position at which the foreign substances are crushed. When the front end of the insertion tube 210 is moved to the target position, the operator discharges the moving crusher 220 out of the insertion tube 210 using the torque piece 225. As a result, the extruded foreign substance moving net 230 expands.

The user applies tension to the pressure wire 250, connected to the foreign substance moving net 230, by rotating the handle 265, so as to change the diameter of the introduction net 231 of the foreign substance moving net 230 in consideration of the diameter of the blood vessel A into which the catheter will be inserted. In this state, as the operator moves the moving shaft 221 back and forth, the pressure tip 223 crushes the blood clots and foreign substances B. A lump of crushed foreign substances C is introduced into the introduction net 231 and is then moved to the flow net 233. At this time, since the surface of the flow net 233 is covered with the coating layer c, the lump of crushed foreign substances C is moved through the flow net 233, rather than being discharged out of the flow net 233.

In addition, the lump of crushed foreign substances is discharged into the insertion tube 210 through the discharge net 235, and is drawn by suction force of the suction unit 240 so as to be extracted into the suction tube 213.

The operator continuously applies pressure to the foreign substances B so as to crush the same while moving the moving shaft 221 back and forth, and the foreign substance moving net 230 expands and contracts by repeatedly entering and exiting the insertion tube 210 through the front end of the insertion tube 210 via the forward and rearward movement of the moving shaft 221. Thereby, most of the lump of crushed foreign substances C is extracted out of the blood vessel through the foreign substance moving net 230. As described above, the catheter for the extraction of foreign substances from blood vessels according to the secondly described embodiment of the present invention has a feature in that the shape and diameter of the foreign substance moving net may be changed unlike the catheter for the extraction of foreign substances from blood vessels according to the firstly described exemplary embodiment. The foreign substance moving net is variable in shape and diameter as pressure is applied to the pressure wire, and therefore may be compatible with various sizes of blood vessels.

1. A catheter for the extraction of foreign substances from blood vessels, the catheter comprising:
an insertion tube configured to be inserted into a blood vessel and provided at one side thereof with a branched suction tube;
a moving crusher configured to be inserted into the blood vessel while being received inside the insertion tube, the moving crusher serving to crush foreign substances inside the blood vessel after being discharged out of the insertion tube; and
a foreign substance moving net coupled to a front end of the moving crusher so as to guide the crushed foreign substances into the insertion tube, the foreign substance moving net having a mesh shape, wherein the foreign substance moving net includes an introduction net having an open front end to allow the foreign substances to be introduced thereinto, a flow net connected to the introduction net and configured to provide a passage for movement of the foreign substances, and a discharge net configured to couple the flow net and the moving crusher to each other and to discharge the foreign substances into the insertion tube, and
wherein the flow net is provided at a surface thereof with a coating layer to prevent the foreign substances from being discharged out of the flow net.

2. The catheter according to claim 1, wherein the coating layer is formed of silicon or PTFE.

3. The catheter according to claim 2, wherein the moving crusher includes:
a moving shaft configured to move inside the insertion tube; and
a pressure tip located at a front end of the moving shaft and configured to apply pressure to the foreign substances so as to crush the foreign substances, and wherein the foreign substance moving net is coupled to the moving shaft at a rear end of the pressure tip.

4. The catheter according to claim 3, wherein the discharge net is gradually reduced in diameter in a direction in which the discharge net is coupled to the moving shaft, and guides the foreign substances so as to be discharged between the moving shaft and the insertion tube.

5. A catheter for the extraction of foreign substances from blood vessels, the catheter comprising:
an insertion tube configured to be inserted into a blood vessel and provided at one side thereof with a branched foreign substance discharge tube;
a moving crusher configured to be inserted into the blood vessel while being received inside the insertion tube, the moving crusher serving to crush foreign substances inside the blood vessel after being discharged out of the insertion tube;
a foreign substance moving net coupled to a front end of the moving crusher so as to guide the crushed foreign substances into the insertion tube, the foreign substance moving net having a tubal shape such that a diameter thereof is gradually reduced with increasing distance from an open front end thereof and decreasing distance to a rear end thereof coupled to the moving crusher; and
a pressure wire having one end secured to an inner sidewall of the front end of the foreign substance moving net and the other end extending outward from the foreign substance moving net so as to change the shape of the foreign substance moving net by applying pressure to the front end of the foreign substance moving net, wherein the foreign substance moving net includes an introduction net having an open front end to allow the foreign substances to be introduced thereinto, a flow net connected to the introduction net and configured to provide a passage for movement of the foreign substances, and a discharge net configured to couple the flow net and the moving crusher to each other and to discharge the foreign substances into the insertion tube, and wherein the flow net is provided at a surface thereof with a coating layer to prevent the foreign substances from being discharged out of the flow net.

6. The catheter according to claim 5, wherein the moving crusher includes:
a moving shaft configured to move inside the insertion tube; and
a pressure tip located at a front end of the moving shaft and configured to apply pressure to the foreign substances so as to crush the foreign substances, and wherein the foreign substance moving net is coupled to the moving shaft at a rear end of the pressure tip.

7. The catheter according to claim 6, further comprising a wire winder, on which the other end of the wire is wound such that a user applies pressure to a front end of the wire by winding or unwinding the wire.
